(19) United States
(12) Patent Application Publication
(10) Pub. No.: US 2003/0087256 A1
(43) Pub. Date: May 8, 2003

(54) Thiophosphate nucleic acid-based compounds

(75) Inventor: Radhakrishnan P. Iyer, Shrewsbury, MA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Micrologix Biotech Inc., Vancouver (CA)

(21) Appl. No.: 10/118,480

(22) Filed: Apr. 8, 2002

Related U.S. Application Data
(60) Provisional application No. 60/282,098, filed on Apr. 6, 2001.

(51) Int. Cl. C12Q 1/68; C07H 21/04; C07H 19/04; G01N 33/53

(52) U.S. Cl. 435/6; 435/7.1; 536/23.1; 536/25.3; 536/26.1

(57) ABSTRACT

The invention comprises short, thiophosphate nucleic acids (primarily mono-, di-, and tri-nucleotides), libraries comprising them, and methods of using them as therapeutic anti-viral (particularly anti-HBV) agents.

This application claims the benefit of U.S. Provisional Application No. 60/282,098, filed Apr. 6, 2001.

### BACKGROUND OF THE INVENTION

1. Field of the Invention

The present invention relates to novel thiophosphorylated nucleic acid-based compounds and libraries containing such compounds. Compounds of the invention will be useful in a variety of applications, e.g. as a nucleoside or oligonucleotide therapeutic agent, or for diagnostic or analytical applications, e.g. as a capture probe in a hybridization assay.

2. Background

A variety of nucleic-acid based compounds have been investigated for therapeutic applications as well as for a range of analytical methods, including as capture probes in hybridization assays. See, for instance, U.S. Pat. No. 5,736,316.

The important initial step in the development of therapeutic agents is the discovery of compounds that bind to a protein, enzyme or receptor of interest. Through careful structure/activity work of resulting active compounds, one arrives at a lead compound for further development into a clinical candidate. That traditional process of drug discovery can be a long and arduous endeavor. Often it takes 10 to 15 years before a new drug makes it into the marketplace.

Certain more recent approaches to the discovery of therapeutics have been developed. In one more modem approach, large libraries of diverse compounds are synthesized and subjected to high throughput screening against a particular molecular target implicated in a disease.

### SUMMARY OF THE INVENTION

We now provide nucleotide-based compounds and libraries that comprise thiophosphate linkages.

Compounds of the invention may comprise a thiophosphate linkage at a variety of positions. Generally preferred are compounds comprising a nucleoside group having a 3′-thiophosphate linkage. Compounds containing a nucleoside group having a 5′-thiophosphate linkage also are preferred.

Compounds of the invention are especially useful as therapeutic agents, particularly antiviral agents. In particular, we have now found that thiophosphate compounds of the invention can exhibit against hepatitis B virus (HBV).

The invention also includes thiophosphate compounds that are covalently linker, particularly via an unsaturated linker group. Preferred linker groups include aromatic or conjugated systems that comprise hydroxy and alkylhydroxy substituents that can facilitate electron transfer and thereby decoupling of the compound from a solid support.

The invention also includes thiophosphate compounds covalently linked to a solid structure, particularly a reaction body, for use in a wide variety of diagnostic and other analytical applications. In particular, a thiophosphate oligonucleotide can be covalently linked to a substrate surface, such as a glass reaction surface, and used as a capture probe in analysis of genetic material. In this aspect, the invention include microarray platforms that comprise a thiophosphate oligonucleotide of the invention, preferably covalently linked to a reaction space of the microarray.

The invention further includes methods for synthesis of nucleotide-based compounds and new libraries of such compounds.

Other aspects of the invention are discussed infra.

### DETAILED DESCRIPTION OF THE INVENTION

As discussed above, thiophosphate compounds and libraries of such compounds are provided.

Preferred thiophosphate compounds of the invention include those of the following Formula I, II, III and IV:

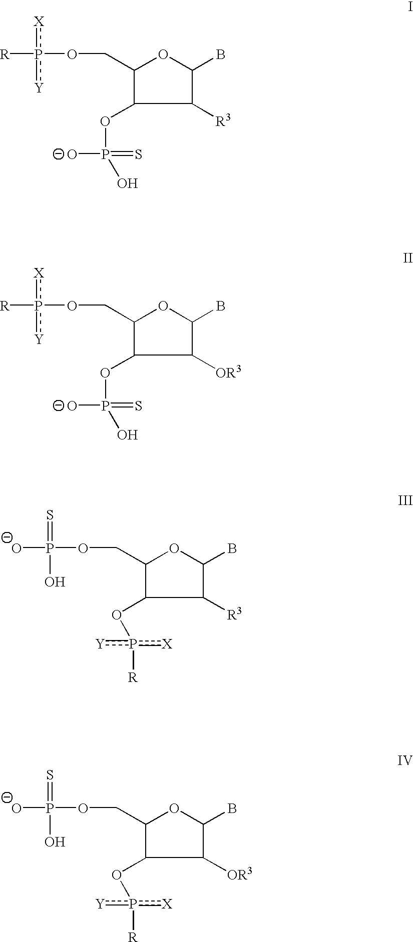

wherein in each of Formulae I, II, III and IV: X and Y is are each independently O, S, Se, NR¹NR², CR¹CR², OR, SR, SeR, or an enzymatically reactive (particularly, cleavable) moiety such as an amide, ester and the like;

R is hydrogen or a hydrophobic group, e.g. a moiety having from 1 to about 18 carbon atoms, such as optionally substituted alkyl, optionally substituted alkenyl, optionally substituted alkynyl, optionally substituted aralkyl, optionally substituted cycloalkyl, optionally substituted cycloalkenyl, optionally substituted carbocyclic aryl, an optionally substituted mononucleotide, an optionally substituted polynucleotide, or an optionally substituted heteroaromatic or heteroalicyclic group preferably having from 1 to 3 separate or fused rings and 1 to 3 N, O or S atoms;

R¹, R² and R³ are each independently selected from a group as defined by R;

B is a base, preferably optionally substituted adenine, optionally substituted thymidine, optionally substituted cytosine or optionally substituted guanine, preferably where the optional substituents are alkyl, carbocyclic aryl, or heteroaromatic or heteroalicyclic group preferably having from 1 to 3 separate or fused rings and 1 to 3 N, O or S atoms, or a heteroalicyclic structure that is covalently linked to the sugar ring;

and pharmaceutically acceptable salts thereof.

Preferred compounds of the invention include those of the following Formulae V, VI, VII and VII, having the depicted configurations:

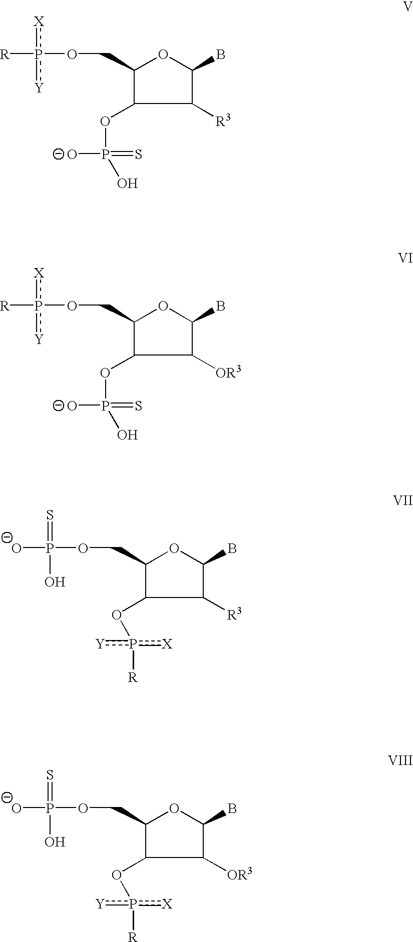

wherein in those Formula V, VI, VII and VIII, X and Y are each independently selected from a group consisting of O, S, Se, NR¹NR², CR¹CR², OR, SR and SeR, or one or both of X and Y are an enzymatically reactive (particularly, cleavable) moiety such as an amide, ester, and the like, with at least one of X and Y being S;

R is hydrogen or a hydrophobic group, e.g. a moiety having from 1 to about 18 carbon atoms, such as optionally substituted alkyl, optionally substituted alkenyl, optionally substituted alkynyl, optionally substituted aralkyl, optionally substituted cycloalkyl, optionally substituted cycloalkenyl, optionally substituted carbocyclic aryl, an optionally substituted mononucleotide, an optionally substituted polynucleotide, or an optionally substituted heteroaromatic or heteroalicyclic group preferably having from 1 to 3 separate or fused ring and 1 to 3 N, O or S atoms;

R¹, R² and R³ are each independently selected from a group as defined by R;

B is is a base, optionally substituted adenine, optionally substituted thymidine, optionally substituted cytosine or an optionally substituted guanine, preferably where the optional substituents are alkyl, carbocyclic aryl, or heteroaromatic or heteroalicyclic group preferably having from 1 to 3 separate or fused rings and 1 to 3 N, O or S atoms, or a heterocyclic structure that is covalently linked to the sugar ring;

and pharmaceutically acceptable salts thereof.

In the above formulae, it is understood that the dashed line extending to each of the substituents X and Y designates that one, but not both, of X and Y may have an additional chemical bond (i.e. to form a double bond).

The depicted sugar group may be natural or modified (e.g. synthetic) form, or in an open chain form (where one of the depicted ring bonds would not be present).

Preferred R groups of compounds of the above formulae include cyclic groups, particularly alicyclic groups that may comprise one or more single or polycyclic rings, particularly a bridged or fused ring structure, with 0, 1 or 2 endocyclic carbon-carbon double bonds. Additional preferred R groups include heteroalicyclic moieties, particularly heteroalicylic.

Preferred compounds of the invention include those of the above formula where the nucleoside is linked to the R group via a phosphorous group at the 5′ end. Such linkages could also be established via the 2′ or 3′ sites of the nucleoside. When R is a nucleoside, linkages can be via 5′ to 3′, 5′ to 5′, 3′ to 3′, 2′ to 5′ and 2′ to 2′, or any combination thereof, of the participating nucleosides.

The depicted sugar group may be natural or modified (e.g. synthetic) form, or in an open chain form (where one of the depicted ring bonds would not be present).

Preferred R groups of compounds of the above include cyclic groups, particularly alicyclic groups that may comprise one or more single or polycyclic rings, particularly a bridged or fused ring structure, with 0, 1 or 2 endocyclic carbon-carbon double bonds. Additional preferred R groups include heteroalicyclic moieties, particularly heteroalicyclic groups having from 5 to about 8 ring member, preferably with one or two O, N or S ring members, particularly one or two oxygen ring members.

As mentioned above, X and Y of the above formulae may be an enzymatically reactive group, i.e. the group may be cleavable or otherwise reactive in vivo upon administration to a mammal, particularly a human. Preferred enzymatically reactive groups include e.g. amides (which may be cleaved in vivo with an amidase), esters (which may be cleaved in vivo with an esterase), and acetal and ketal groups.

Preferred compounds of the invention include those of the above formula where the nucleoside is linked to the R group via a phosphorous group at the 5′ end. Such linkages could also be established via the 2′ or 3′ sites of the nucleoside. When R is a nucleoside, linkages can be via 5′ to 3′, 5′ to 5′, 3′ to 3′, 2′ to 5′ and 2′ to 2′, or any combination thereof, of the participating nucleosides.

Preferably, compounds of the invention will be present in enantiomerically enriched mixtures, i.e. where one enantiomer is present in a greater amount than other stereoisomer(s) of the compound, particularly where one enantiomer is present in amount of at least about 60 mole percent, relative to all stereoisomers present of the compound; preferably where one enantiomer is present in amount of at least about 70 or 80 mole percent, relative to all stereoisomers present of the compound; still more preferably where one enantiomer is present in amount of at least about 85, 90, 92, 95, 96, 97, 98 or 99 mole percent, relative to all stereoisomers present of the compound.

In the above formulae, alkyl groups preferably contain from 1 to about 18 carbon atoms, more preferably from 1 to about 12 carbon atoms and most preferably from 1 to about 6 carbon atoms. Specific examples of alkyl groups include, for example, methyl, ethyl, n-propyl, isopropyl, n-butyl, t-butyl, isobutyl, pentyl, hexyl, heptyl, octyl, nonyl, decyl, etc.

In the above formulae, aralkyl groups include the above-listed alkyl groups substituted by a carbocyclic aryl group having 6 or more carbons, for example, phenyl, naphthyl, phenanthryl, anthracyl, etc.

In the above formulae, cycloalkyl groups preferably have from 3 to about 8 ring carbon atoms, e.g. cyclopropyl, cyclopentyl, cyclohexyl, cycloheptyl, 1,4-methylenecyclohexane, adamantyl, cyclopentylmethyl, cyclohexylmethyl, 1- or 2-cyclohexylethyl and 1-, 2- or 3-cyclohexylpropyl, etc.

In the above formulae, exemplary heteroaromatic and heteroalicyclic group include pyridyl, pyrazinyl, pyrimidyl, furyl, pyrrolyl, thienyl, thiazolyl, oxazolyl, imidazolyl, indolyl, benzothiazolyl, tetrahydrofuranyl, tetrahydropyranyl, piperidinyl, morpholino and pyrrolidinyl.

Mononucleotides of compounds of the invention invention include adenine, cytodine, guanosine and thymidine.

Polynucleotides of compounds of the invention preferably contain from about 1 to about 20 mononuculeotides, more preferably from 1 to about 10 mononuculeotides and still more preferably from 1 to about 5 mononuculeotides. The polynucleotides are suitably constructed such that the 5′ group of one mononucleotide pentose ring is attached to the 3′ group of its neighbor in one direction via, for example, a phosphodiester or a phosphorthioate internucleotide linkage.

Sugar groups of compounds of the invention may be comprised of mono-, di-, oligo- or poly-saccharides wherein each monosaccharide unit comprises from 3 to about 8 carbons, preferably from 3 to about 6 carbons, containing polyhydroxy groups or polyhydroxy and amino groups. Non-limiting examples include glycerol, ribose, fructose, glucose, glucosamine, mannose, galactose, maltose, cellobiose, sucrose, starch, amylose, amylopectin, glycogen and cellulose. The hydroxyl and amino groups are present as free or protected groups containing e.g. hydrogens and/or halogens. Preferred protecting groups include acetonide, t-butoxy carbonyl groups, etc. Monosaccharide sugar groups may be of the L or D configuration and a cyclic monosaccharide unit may contain a 5 or 6 membered ring of the α or β conformation. Disaccharides may be comprised of two identical or two dissimilar monosaccharide units. Oligosaccharides may be comprised of from 2 to 10 monosaccharides and may be homopolymers, heteropolymers or cyclic polysugars. Polysaccharides may be homoglycans or heteroglycans and may be branched or unbranched polymeric chains. The di-, oligo- and poly-saccharides may be comprised of 1→4, 1→6 or a mixture of 1→4 and 1→6 linkages. The sugar moiety may be attached to the link group through any of the hydroxyl or amino groups of the carbohydrate.

The invention includes novel linker groups that are particularly useful for linking a nucleic-acid based compound, particularly a thiophosphate compound, to a solid support such as a glass or polymer substrate. The linker group preferably is aromatic or otherwise has multiple bonds that can facilitate electron transfer and is substituted by at least one hydroxy or amino group and at least one alkylamino or alkylhydroxy group, such as C₁₋₈alkylamino or C₁₋₈alkylhydroxy, particularly —CH₂NH₂ and —CH₂OH. The multiple bond moiety of the linker may be e.g. a single or fused ring compound such as phenyl, naphthyl and the like, or separate linked rings such as bi-phenyl that can enable electron transfer, or a non-aromatic conjugated system. For instance, suitable linkers include the following compounds A through F. Scheme 1 below also exemplifies a linker group and deprotection reaction in accordance with the invention. In those structures A through F below, X and Y each represent a carbon or hetero atom such O, S or N, and the group M represent one or more non-hydrogen ring substituents such as halo, or a group as defined for R.

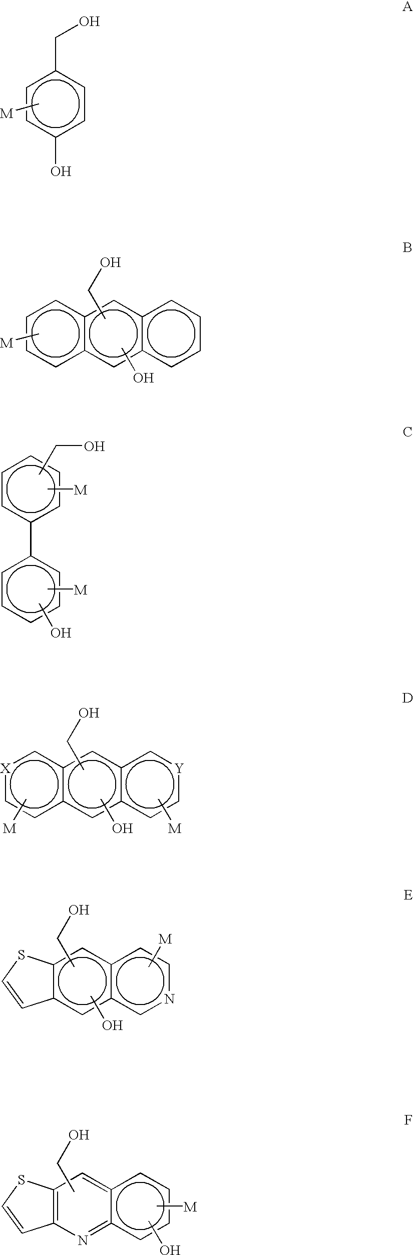

In view of the foregoing, the following embodiments of compounds, libraries, compositions, and methods are preferred. A compound comprising a structure of the following formula:

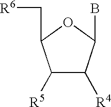

and pharmaceutically acceptable salts thereof, wherein

R⁴ is —R³ or —OR³;

one of R⁵ and R⁶ is

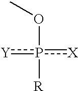

and the other of R⁵ and R⁶ is

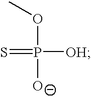

X and Y are independently O, S, Se, NR¹, NR¹NR². CR¹CR², OR⁷, SR⁷, and SeR⁷, provided at least one of X and Y is S;

R is —OH, a mononucleoside, or a dinucleotide;

R¹, R², R³, and R⁷ are independently H or a C₁-C₂₀ hydrophobic moiety; and

B is a purine or pyrimidine base.

Preferably, the compound has the following stereochemistry:

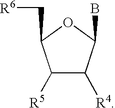

In one preferred embodiment, R, R¹, R², R³, and R⁷ independently are —H; —OH; C₁-C₂₀ straight, branched, or cyclic alkyl; C₂-C₂₀ straight, branched, or cyclic alkenyl; C₂-C₂₀ straight or branched alkynyl; C₅-C₂₀ heterocyclyl having from 1 to 3 separate or fused rings and 1 to 3 N, O, or S atoms, or C₅-C₂₀ heteroaryl having from 1 to 3 separate or fused rings and 1 to 3 N, O, or S atoms, provided that annular O and S atoms are not covalently bound to another annular O or S.

The heterocyclyl and heteroaryl groups are preferably selected from pyridyl, pyrazinyl, pyrimidyl, furyl, pyrrolyl, thienyl, thiazolyl, oxazolyl, imidazolyl, indolyl, benzothiazolyl, tetrahydrofuranyl, tetrahydropyranyl, piperidinyl, morpholino, and pyrrolidinyl.

B is preferably adenine, thymidine, cytosine, or guanine, each of which is optionally substituted by C₁-C₆-alkyl C₁-C₂₀ straight, branched, or cyclic alkyl; C₂-C₂₀ straight, branched, or cyclic alkenyl; C₂-C₂₀ straight or branched alkynyl; C₅-C₂₀ heterocyclyl having from 1 to 3 separate or fused rings and 1 to 3 N, O, or S atoms, or C₅-C₂₀ heteroaryl having from 1 to 3 separate or fused rings and 1 to 3 N, O, or S atoms.

In one preferred embodiment, the compound is a mononucleotide; in another it is a dinucleotide.

In another aspect, the invention comprises a library comprising 2 or more different compounds, each as described above. Preferably, the library comprises 20 or more different compounds. Also preferably, the compounds are mononucleotides or dinucleotides.

In one embodiment, the compounds of the library are covalently bound to a solid surface via a linker moiety. Preferably the solid support is a microarray substrate. The linker moiety is preferably a structure of formula

—Z—A—Z′—

wherein,

one of Z and Z′ is —O— or —NH— and the other is —O—C₁₋₆-alkyl or —NH—C₁₋₆-alkyl; and

A is an aromatic moiety or a moiety having one or more multiple bonds that can facilitate electron transfer, and wherein said moiety is optionally substituted by —OH; C₁-C₂₀ straight, branched, or cyclic alkyl; C₂-C₂₀ straight, branched, or cyclic alkenyl; C₂-C₂₀ straight or branched alkynyl; C₅-C₂₀ heterocyclyl having from 1 to 3 separate or fused rings and 1 to 3 N, O, or S atoms, or C₅-C₂₀ heteroaryl having from 1 to 3 separate or fused rings and 1 to 3 N, O, or S atoms.

Preferably A is an optionally substituted di-radical of benzene, naphthalene, phenylbenzene, and anthracene. More preferably, A is a di-radical of benzene, naphthalene, phenylbenzene, and anthracene.

Preferred linkers of structure —Z—A—Z′— are selected from selected from the group consisting of:

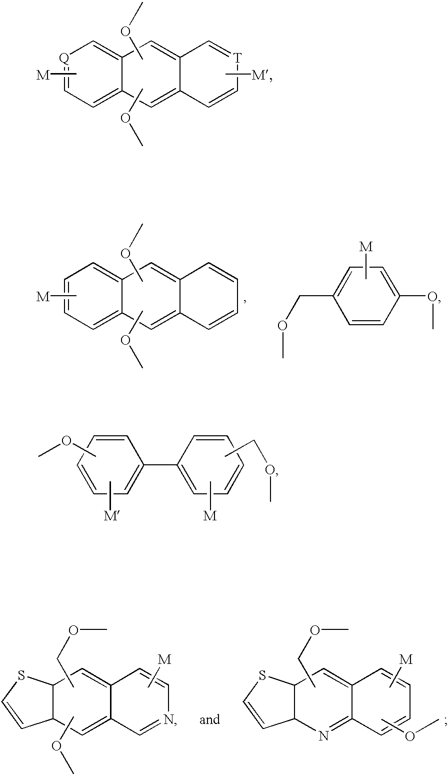

and wherein M is halo, C₁-C₂₀ straight, branched, or cyclic alkyl; C₂-C₂₀ straight, branched, or cyclic alkenyl; C₂-C₂₀ straight or branched alkynyl; C₅-C₂₀ heterocyclyl having from 1 to 3 separate or fused rings and 1 to 3 N, O, or S atoms, or C₅-C₂₀ heteroaryl having from 1 to 3 separate or fused rings and 1 to 3 N, O, or S atoms.

In another aspect, the invention comprises a composition comprising a compound as described above and a pharmaceutically acceptable carrier. In another aspect, the invention comprises a method of treating a subject suffering from or susceptible to HBV comprising administering to the subject an effective amount of such a composition.

Compounds of the invention may be suitably prepared as depicted in the following Scheme I. Linkers are employed in the solid-phase synthesis of a library of 3′-thiophosphorlayetd dinucleotides 3 as shown in Scheme 1.

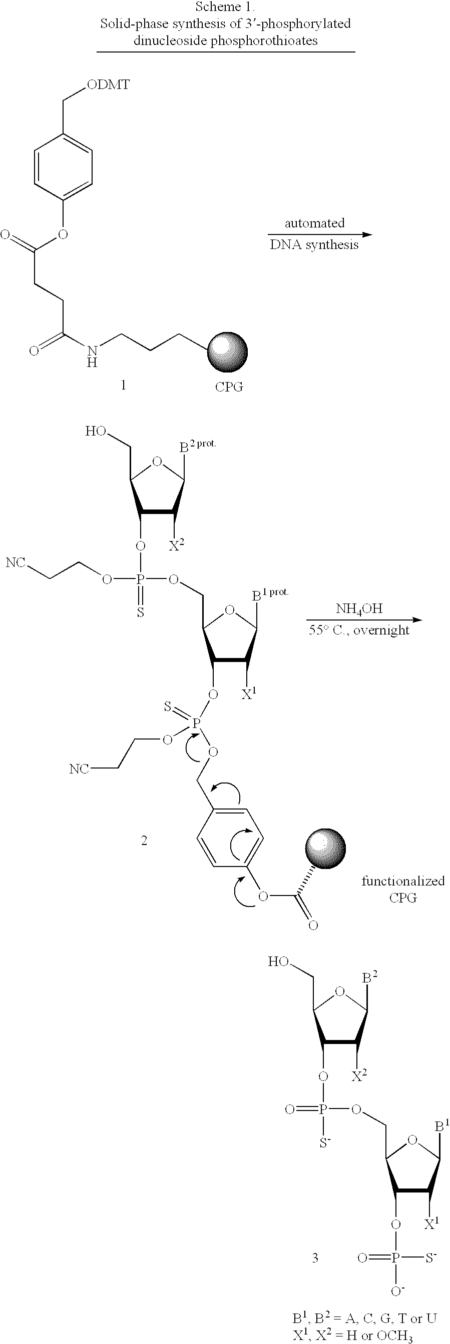

The support-bound acyloxyaryl derivative 1 was prepared as shown in Scheme 2 below. Thus, treatment of the aminoalkyl-CPG 4 with succinic anhydride (DMAP/pyridine, 24 h) led to the succinylated-CPG 5. Reaction of the derivatized CPG 5 with the alcohol 6 (DMAP/EDC/TEA/pyridine, 48 h) gave the support-bound derivative 1. The loading of the linker on the support was estimated to be in the range of 60 to 90 μmol/g, based upon the 4′,4′-dimethoxytrityl cation assay.

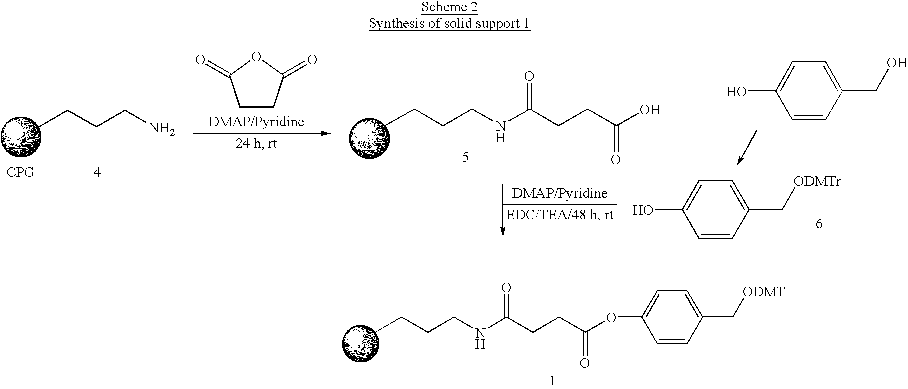

Synthesis of library 3 can be suitably initiated in an automated DNA synthesizer on a 15 μmol scale (DMT-off), using two cycles of the standard DNA synthesis protocol that employed phosphoramidite chemistry (Scheme 3 below). Thus, the deprotection of the 4′, 4′-dimethoxytrityl group 1 followed by coupling with the first nucleoside phosphoramidite 8 yields the phosphate triester 9 (B=A, C, G, T or U; X=H or OCH3). Oxidiative sulfuriation of 9 with 3H-benzodithiole-3-one-1,1-dioxide leads to the thiophosphotriester 10 which can be carried through a second round of the synthesis cycle to provide the support-bound dinucleotide 2. Additional rounds through the synthesis cycle with provide higher order oligonucleotides.

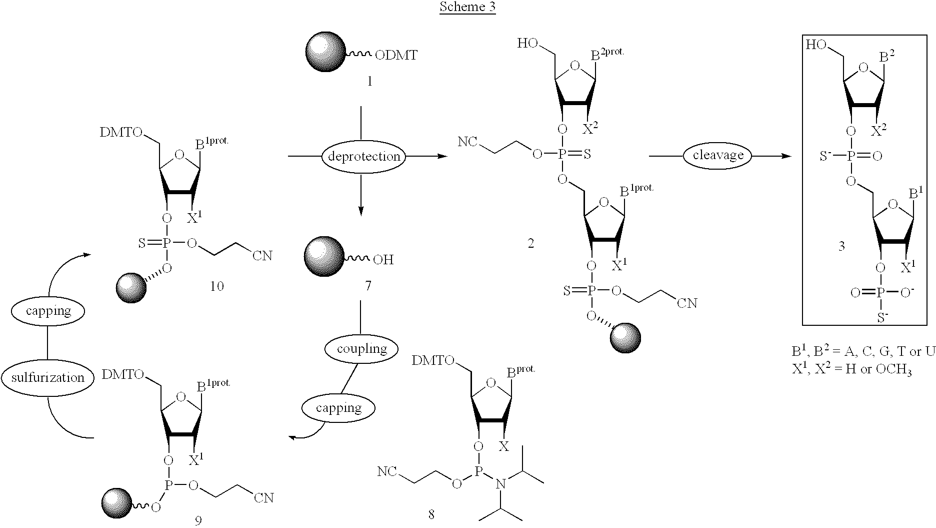

Deprotection as shown in Scheme 3 above can be accomplished by treatment acid such as 5% dichloroacetic acid in dichloromethane; coupling: 1H-tetrazole in acetonitrile; capping: acetic anhydride and N-methylimidazole in THF; Sulfurization: 3H-1,2-benzodithiole-3-one-1,1-dioxide in CH₃CN; cleavage: treatment with base such as 28% aq. NH₄OH, 55° C., overnight

The cleavage of the solid-support and the deprotection of nucleobases and thiophosphate groups were conducted in a single step by treatment with concentrated ammonium hydroxide. The mechanism of cleavage of the acyloxyaryl group is postulated in Scheme 1 above where the first thiophosphate triester group linked to the solid-support acts as an effective leaving group releasing the 3′-thiophosphorylated dinucleotide.

A sixty-four member library 3 representing 3′-psXY dimer sequences (X,Y=dA, dC, dG, dT, 2′-OMe-rA, 2′-OMe-rC, 2′-OMe-rG or 2′-OMe-rU) was prepared using this approach. Purification of the crude library members was achieved by passing through a weak anion-exchange resin (DEAE-5PW) followed by a desalting chromatography on C₁₈ column. These sequential steps removed the by-products such as benzylamide, isobutyrylamide resulting from the base deprotection, as well as, the unreacted 3′-thiophosphate monomer 7. The purity of the resulting library members, determined by HPLC, ranged between 85% and 99%. The average yield of the library members was 50 to 55%. ³¹P, ¹H NMR, and MS analysis of selected library members confirmed their identity.

Compound libraries of the invention preferably will contain at least about 2, 3, 4 or 5 distinct compounds, more preferably at least about 10 distinct compounds, still more preferably at least about 20, 30, 40, 50 ,60, 70, 80, 90 or 100 compounds, and may contain 200, 300, 400 or 500 or more compounds.

Compounds of the invention will be useful for a variety or therapeutic application, including in methods of treatment against infections and diseases associated with a virus, particularly a hepadnavirus such as HBV. The invention thus includes methods of treatment of a mammal susceptible to (prophylactic treatment) or suffering from a disease associated with a virus, particularly a hepadnavirus, especially hepatitis B (HBV) virus. Methods of the invention generally include administration to a mammal, particularly a primate such as a human, in need of treatment a therapeutically effective amount of one or more compounds of the invention.

Compounds of the invention may be used as inhibitors of viral kinases, viral polymerases, and as disrupters of helicase-primase complexes with nucleic acids during viral replication.

Administration of compounds of the invention may be made by a variety of suitable routes including oral, topical (including transdermal, buccal or sublingal), nasal and parenteral (including intraperitoneal, subcutaneous, intravenous, intradermal or intramuscular injection) with oral or parenteral being generally preferred. It also will be appreciated that the preferred method of administration and dosage amount may vary with, for example, the condition and age of the recipient.

Compounds of the invention may be used in therapy in conjunction with other pharmaceutically active medicaments, such as another anti-viral agent, or an anti-cancer agent. Additionally, while one or more compounds of the invention may be administered alone, they also may be present as part of a pharmaceutical composition in mixture with conventional excipient, i.e., pharmaceutically acceptable organic or inorganic carrier substances suitable for parenteral, oral or other desired administration and which do not deleteriously react with the active compounds and are not deleterious to the recipient thereof. Suitable pharmaceutically acceptable carriers include but are not limited to water, salt solutions, alcohol, vegetable oils, polyethylene glycols, gelatin, lactose, amylose, magnesium stearate, talc, silicic acid, viscous paraffin, perfume oil, fatty acid monoglycerides and diglycerides, petroethral fatty acid esters, hydroxymethyl-cellulose, polyvinylpyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously react with the active compounds.

For parenteral application, particularly suitable are solutions, preferably oily or aqueous solutions as well as suspensions, emulsions, or implants, including suppositories. Ampules are convenient unit dosages.

For enteral application, particularly suitable are tablets, dragees or capsules having talc and/or carbohydrate carrier binder or the like, the carrier preferably being lactose and/or corn starch and/or potato starch. A syrup, elixir or the like can be used wherein a sweetened vehicle is employed. Sustained release compositions can be formulated including those wherein the active component is protected with differentially degradable coatings, e.g., by microencapsulation, multiple coatings, etc.

Therapeutic compounds of the invention also may be incorporated into liposomes. The incorporation can be carried out according to known liposome preparation procedures, e.g. sonication and extrusion. Suitable conventional methods of liposome preparation are also disclosed in e.g. A. D. Bangham et al., J. Mol. Biol., 23:238-252 (1965); F. Olson et al., Biochim. Biophys. Acta, 557:9-23 (1979); F. Szoka et al., Proc. Nat. Acad. Sci., 75:4194-4198 (1978); S. Kim et al., Biochim. Biophys. Acta, 728:339-348 (1983); and Mayer et al., Biochim. Biophys. Acta, 858:161-168 (1986).

It will be appreciated that the actual preferred amounts of active compounds used in a given therapy will vary according to the specific compound being utilized, the particular compositions formulated, the mode of application, the particular site of administration, etc. Optimal administration rates for a given protocol of administration can be readily ascertained by those skilled in the art using conventional dosage determination tests.

As discussed above, compounds of the invention also may be used in diagnostic and other analytical methods, particularly in array platforms where a thiophosphate linkage is covalently attached to the array platform such as a glass slide. The linked compound may be e.g. an oligonucleotide having from 2 to about 100 residues, more typically about 4 to about 10, 15, 20, 25, 30, 35, 40, 45, 50, 60, 70 or 80 residues. In use, a test sample, e.g. a patient's fluid sample (e.g. blood sample) for a diagnostic application, can be applied to the linked oligonucleotide on the reaction body (e.g. glass slide) and desired hybridization detected.

All documents are incorporated herein by reference.

The following examples are illustrative of the invention. The material reference numbers in the following samples are the same as designated in Schemes 1 through 3 above.

### EXAMPLE 1

### Synthesis of the Solid-support 1

To a solution of 4-hydroxybenzyl alcohol (1.24 g, 10 mmol) in anhydrous pyridine (30 mL) was added 4,4′-dimethoxytritylchloride (3.38 g, 10 mmol). The mixture was stirred overnight. Pyridine was evaporated under reduced vacuum. Dichloromethane (20 mL) was added and the organic layer was washed with water (3×10 mL) and dried over anhydrous MgSO₄. After evaporation of the solvent, the residue was purified by flash chromatography on silica gel (hexane:ethyl acetate=2:1) to give 3.89 g of the product 6 as a yellow solid in 89% yield. ¹H NMR (CDCl₃, 500 MHz, ppm) 3.79 (s, OCH₃, 6H), 4.07 (s, OCH₂,Ar, 2H), 4.72 (br, OH, 1H), 6.80 (d, J=8.5 Hz, H—Ar—OH, 2H), 6.83 (d, J=8.7 Hz, H—Ar—OCH₃, 4H), 7.22 (d, J=7.6 Hz, H—Ar, 1H), 7.24 (d, J=8.5 Hz, H—Ar—OH, 2H), 7.29 (t, J=7.6 Hz, H—Ar, 2H), 7.40 (d, J=8.7 Hz, H—Ar—OCH₃, 4H), 7.49 (d, J=7.6 Hz, H—Ar, 2H).

The ether 6 was employed in the synthesis of the solid-support 1 as follows:

(a) Functionalization of the aminopropyl-CPG 4: Succinic anhydride (20 mmol, 2 g) and DMAP (2 mmol, 244 mg) were added to oven-dried aminopropyl-CPG 4 (10 g) with 80 mL of anhydrous pyridine. The mixture was shaken at room temperature for 24 h. The succinylated CPG S was filtered and washed with pyridine (2×50 mL) and dichloromethane (3×50 mL).

(b) Anchorage of the linker: The aralkyl ether 6 (2 mmol, 872 mg) and DMAP (2 mmol, 244 mg) were coevaporated twice with anhydrous pyridine and dried overnight under high vacuum. To this mixture, dissolved in anhydrous pyridine (100 mL), were added the succinylated CPG 5, 1-(3-dimethylaminopropyl)-3-ethylcarbodiimide hydrochloride, (20 mmol, 3.8 g), and triethylamine (0.8 mL) and then shaken for 48 h.

(c) Capping steps: Pentachlorophenol (5 mmol, 1.35 g) was then added to the mixture which was shaken for additional 15 h. Piperidine (50 mL) was then added and stirring was applied for only 5 minutes before collecting the resin by filtration and washed it with dichloromethane (4×100 mL) and ether (2×100 mL). The dry solid-support was mixed with Cap A (contents: THF/Ac₂O 9:1) (50 mL) and Cap B (contents: 10% N-methylimidazole in THF/pyridine 8:1) (50 mL) for another one hour. CPG was collected by filtration and washed with dichloromethane (3×200 mL) and ether (2×200 mL). Finally, the CPG was dried overnight under high vaccum prior to use in synthesis.

(d) 4,4′-dimethoxytrityl cation assay procedure: To a solution of 14 mg of dry solid-support 1 in dichloromethane (70 mL) was added 0.2 mL of perchloric acid. The mixture was slowly stirred for 20 minutes. UV absorbance was measured at 503 nm and the loading was determined.

### EXAMPLE 2

### Assembly of the Library 3

The library synthesis was performed on a 15 μmol scale using the standard automated DNA synthesis protocol (DMT-off) used for the oligonucleoside phosphorothioate assembly. After synthesis, the CPG was dried in the column and transferred to 5 mL safe-sealed polypropylene tubes for ammonium hydroxide (28%, 4 mL, 55° C., overnight). After cooling down, the solution was separated from the CPG and ammonium hydroxide was evaporated. The resulting aqueous solution was analyzed by HPLC. The samples showing more than 85% purity were submitted to ethyl acetate extraction (2×1 mL) while the others were purified by anion-exchange resin (DEAE-5PW, gradient of 0.5 M NaCl in H₂O from 0 to 40%) followed by desalting on reverse-phase column (C₁₈, Buffer A: H₂O, Buffer B: 20%CH₃CN in H₂O). In both cases, the residual organic solvents were evaporated before filtration through 0.2 μm filter. Each product appeared as a white foam after lyophilization. The structure of the selected library members was determined by NMR and mass spectra. ³¹P NMR analysis revealed clear signals of the triester phosphorothioate (doublet ca. 58 and 61 ppm) and diester phosphorothioate (single peak ca. 47 ppm). ¹H NMR and MS were in agreement with the expected structures.

The foregoing description of the invention is merely illustrative thereof, and it is understood that variations and modifications can be effected without departing from the spirit or scope of the invention as set forth in the following claims.

1. A compound comprising a structure of the following formula: 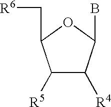
and pharmaceutically acceptable salts thereof, wherein
R⁴ is —R³ or —OR³;
one of R⁵ and R⁶ is 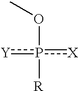
and the other of R⁵ and R⁶ is 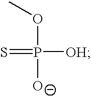
X and Y are independently O, S, Se, NR¹, NR¹NR². CR¹CR², OR⁷, SR⁷, and SeR⁷, provided at least one of X and Y is S;
R is —OH, a mononucleoside, or dinucleotide;
R¹, R², R³, and R⁷ are independently H or a C₁-C₂₀ hydrophobic moiety; and
B is a purine or pyrimidine base:

2. The compound of claim 1 having the following stereochemistry:

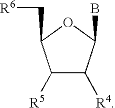

3. The compound of claim 1 wherein R, R¹, R², R³, and R⁷ independently are —H; —OH; C₁-C₂₀ straight, branched, or cyclic alkyl; C₂-C₂₀ straight, branched, or cyclic alkenyl; C₂-C₂₀ straight or branched alkynyl; C₅-C₂₀ heterocyclyl having from 1 to 3 separate or fused rings and 1 to 3 N, O, or S atoms, or C₅-C₂₀ heteroaryl having from 1 to 3 separate or fused rings and 1 to 3 N, O, or S atoms, provided that annular O and S atoms are not covalently bound to another annular O or S.

4. The compound according to claim 3 wherein the heterocyclyl and heteroaryl groups are selected from pyridyl, pyrazinyl, pyrimidyl, furyl, pyrrolyl, thienyl, thiazolyl, oxazolyl, imidazolyl, indolyl, benzothiazolyl, tetrahydrofuranyl, tetrahydropyranyl, piperidinyl, morpholino, and pyrrolidinyl.

5. The compound of claim 1, wherein B is adenine, thymidine, cytosine, or guanine, each of which is optionally substituted by C₁-C₆-alkyl C₁-C₂₀ straight, branched, or cyclic alkyl; C₂-C₂₀ straight, branched, or cyclic alkenyl; C₂-C₂₀ straight or branched alkynyl; C₅-C₂₀ heterocyclyl having from 1 to 3 separate or fused rings and 1 to 3 N, O, or S atoms, or C₅-C₂₀ heteroaryl having from 1 to 3 separate or fused rings and 1 to 3 N, O, or S atoms.

6. The compound of claim 5, wherein the mononucleotide.

7. The compound of claim 5, wherein the compound is a dinucleotide.

8. A library comprising 2 or more different compounds according to claim 1.

9. The library according to claim 8 comprising 20 or more different compounds.

10. The library according to claim 8, wherein the compounds are mononucleotides.

11. The library according to claim 8, wherein the compounds are dinucleotides.

12. The library according to claim 8, wherein the compounds are covalently bound to a solid surface via a linker moiety.

13. The library according to claim 12, wherein the solid support is a microarray substrate.

14. The library according to claim 12, wherein the linker comprises a structure of formula
—Z—A—Z′—
wherein,
one of Z and Z′ is —O— or —NH— and the other is —O—C₁₋₆-alkyl or —NH—C₁₋₆-alkyl; and
A is an aromatic moiety or a moiety having one or more multiple bonds that can facilitate electron transfer, and wherein said moiety is optionally substituted by —OH; C₁-C₂₀ straight, branched, or cyclic alkyl; C₂-C₂₀ straight, branched, or cyclic alkenyl; C₂-C₂₀ straight or branched alkynyl; C₅-C₂₀ heterocyclyl having from 1 to 3 separate or fused rings and 1 to 3 N, O, or S atoms, or C₅-C₂₀ heteroaryl having from 1 to 3 separate or fused rings and 1 to 3 N, O, or S atoms.

15. The library according to claim 14, wherein A is an optionally substituted di-radical of benzene, naphthalene, phenylbenzene, and anthracene.

16. The library according to claim 14, wherein A is a di-radical of benzene, naphthalene, phenylbenzene, and anthracene.

17. The library according to claim 14, wherein —Z—A—Z′— is selected from selected from the group consisting of: 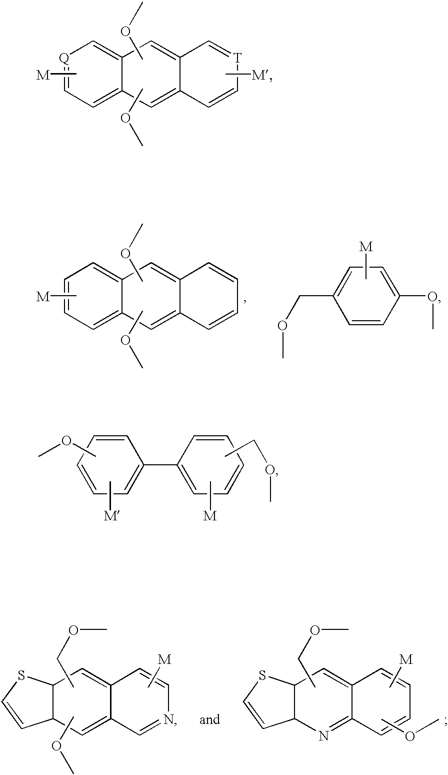
and wherein M is halo, C₁-C₂₀ straight, branched, or cyclic alkyl; C₂-C₂₀ straight, branched, or cyclic alkenyl; C₂-C₂₀ straight or branched alkynyl; C₅-C₂₀ heterocyclyl having from 1 to 3 separate or fused rings and 1 to 3 N, O, or S atoms, or C₅-C₂₀ heteroaryl having from 1 to 3 separate or fused rings and 1 to 3 N, O, or S atoms.

18. A composition comprising a compound according to claim 1 and a pharmaceutically acceptable carrier.

19. A method of treating a subject suffering from or susceptible to HBV comprising administering to the subject an effective amount of a compound according to claim 18.
